(19) United States
(12) Patent Application Publication
(10) Pub. No.: US 2013/0195762 A1
(43) Pub. Date: Aug. 1, 2013

(54) FAT CELLS AND METHODS OF USING THEREOF

(71) Applicant: EMBER THERAPEUTICS, INC., Watertown, MA (US)

(72) Inventor: Louis Anthony Tartaglia, Newton, MA (US)

(73) Assignee: EMBER THERAPEUTICS, INC., Watertown, MA (US)

(21) Appl. No.: 13/750,482

(22) Filed: Jan. 25, 2013

Related U.S. Application Data
(60) Provisional application No. 61/591,919, filed on Jan. 29, 2012.

(51) Int. Cl. C12N 5/071

(52) U.S. Cl. CPC C12N 5/0653
USPC 424/9.2; 435/325; 435/366; 435/354; 435/7.92; 435/6.12

(57) ABSTRACT

Beige cells that contain a reporter gene are disclosed herein. Such cells can be used to identify therapeutic agents for treatment of obesity and its associated disorders, such as diabetes.

### CROSS REFERENCE TO RELATED APPLICATIONS

This application claims priority to U.S. Ser. No. 61/591,919, filed on Jan. 29, 2012. The contents of the aforementioned application are hereby incorporated by reference in their entirety.

### BACKGROUND

Adipose tissue is a major endocrine organ that exerts a profound influence on whole-body energy homoeostasis. Two types of adipose tissue exist in mammals: WAT (white adipose tissue) and BAT (brown adipose tissue). WAT stores energy and is the largest energy reserve in mammals, whereas BAT, expressing UCP1 (uncoupling protein 1), is an organ specifically evolved to safely dissipate energy through the oxidation of glucose and other fuels (Cannon and Nedergaard, Physiol. Rev. 2004; 84: 277-359). Excess accumulation of white fat results in obesity and conditions related to obesity are increasingly common, such as metabolic syndrome, impaired fasting glucose (IFG), and impaired glucose tolerance (IGT), all of which indicate high risk for type 2 diabetes (T2DM). Obesity is also associated with additional diseases including cardiovascular disease, hypertension, and cancer. Obesity is caused by chronic energy imbalance, for example, excessive energy intake, moderate energy expenditure, or both. The epidemic of obesity has increased the interest in searching for effective treatments.

### SUMMARY

This disclosure relates, inter alia, to fat cells (e.g., beige cells) that contain a reporter gene. Such fat cells (e.g., beige cells) can be used to evaluate treatments, e.g., to identify compounds (e.g., therapeutic agents), for treatment of obesity and its associated disorders, such as diabetes. This is, at least in part, based on the findings that modulation of certain biological pathways (e.g., PRDM16 pathway) can augment BAT levels and/or activity (e.g., activate, produce, or enhance the production of cells of the brown fat lineage (e.g., beige cells), e.g., from progenitor cells). BAT can dissipate energy through adaptive thermogenesis. Augmentation of BAT levels and/or activity can result in weight loss as predicted by thermodynamic models of energy expenditure, and therefore has a greater than expected impact on treating or preventing disorders, e.g., metabolic disorders including type II diabetes.

In one aspect, the disclosure features a fat cell (e.g., a brown fat lineage cell (e.g., a beige cell) or a progenitor thereof) comprising a reporter gene, wherein the reporter gene is under the control of, e.g., inserted at, e.g., the transcription of which is initiated from, a genetic locus comprising a gene described herein, e.g., a gene in the PRDM16 pathway, e.g., PRDM16.

In some embodiments, the cell is a brown fat lineage cell (e.g., a beige cell) or a progenitor thereof. In some embodiments, the cell is an animal cell, e.g., a mammalian cell, e.g., a human cell or a mouse cell. In some embodiments, the cell is a cultured cell. In some embodiments, the cell is a primary cell, a non-primary cell, an immortalized cell, or a clonal cell.

In some embodiments, the reporter gene encodes a protein useful for monitoring gene expression, e.g., a protein which luminesces or fluoresces, which is colored or produces a colored substrate or an enzymatic activity, e.g., phosphatase activity. In some embodiments, the reporter gene encodes a protein selected from the group consisting of luciferase (e.g., firefly luciferase and Renilla luciferase), green fluorescent protein (GFP), red fluorescent protein (RFP), yellow fluorescent protein (YFP), chloramphenicol acetyltransferase (CAT), alkaline phosphatase (AP) (e.g., secreted embryonic alkaline phosphatase (SEAP)), β-galactosidase (β-gal), β-lactamase (Bla), horseradish peroxidase (HRP), and a variant thereof.

In some embodiments, the genetic locus comprises a gene in the PRD1-BF1-RIZ1 homologous domain containing 16 (PRDM16) pathway, e.g., PRDM16, PGC-1α, and PGC-1β. In some embodiments, the genetic locus comprises PRDM16 gene.

In some embodiments, the cell is derived (e.g., isolated) from the stromal-vascular fraction (SVF) of a subject.

In another aspect, the disclosure features a method of evaluating a treatment, e.g., a compound, e.g., identifying a therapeutic agent, the method comprising: providing a fat cell described herein, e.g., a brown fat lineage cell, e.g., a beige cell, or a progenitor thereof, comprising a reporter gene (e.g., a reporter gene described herein), wherein the reporter gene is under the control of, e.g., inserted at, e.g., the transcription of which is initiated from, a genetic locus comprising a gene described herein, e.g., a gene in the RPDM16 pathway, e.g., RPDM16; contacting a compound, e.g., a therapeutic agent, with the cell; and determining the activity of the reporter gene expression product, thereby evaluating a treatment.

In some embodiments, the method further comprises selecting a compound, e.g., a therapeutic agent, that increases the activity of the reporter gene expression product by at least about 2, 5, 10, 20, 40, 60, 80, 100, 200, 400, 600, 800, or 1000 fold, compared to a control, e.g., the activity of the reporter gene expression product when the cell does not contact the compound, e.g., the therapeutic agent.

In some embodiments, the method is performed in vitro. In some embodiments, the method is performed in vivo, e.g., using an animal model of obesity and/or diabetes. In some embodiments, the method further comprises retesting the identified treatment. In some embodiments, the method is performed in vitro, and further comprises retesting in vivo, e.g., using an animal model of obesity and/or diabetes.

In some embodiments, the method further comprises evaluating one or more genes, the expression of which is upregulated upon stimulation, e.g., by cold or pathways that elevate cyclic adenosine monophosphate (cAMP). In some embodiments, the gene is selected from the group consisting of UCP1, COX7A1, and CIDEA. In some embodiments, the expression of the gene is increased by at least about 2, 5, 10, 20, 40, 60, 80, 100, 120, 140, 160, 180, 200, 250, 300, 400, 500 fold or more, upon stimulation, e.g., by cold or pathways that elevate cyclic adenosine monophosphate (cAMP).

In yet another aspect, the disclosure features a beige cell comprising a reporter gene, wherein the reporter gene is under the control of, e.g., inserted at, e.g., the transcription of which is initiated from, a genetic locus comprising a gene, the expression of which is upregulated upon stimulation, e.g., by cold or pathways that elevate cyclic adenosine monophosphate (cAMP).

In some embodiments, the cell is an animal cell, e.g., a mammalian cell, e.g., a human cell or a mouse cell. In some embodiments, the cell is a cultured cell. In some embodiments, the cell is a primary cell, a non-primary cell, an immortalized cell, or a clonal cell.

In some embodiments, the reporter gene encodes a protein useful for monitoring gene expression, e.g., a protein which luminesces or fluoresces, which is colored or produces a colored substrate or an enzymatic activity, e.g., phosphatase activity. In some embodiments, the reporter gene encodes a protein selected from the group consisting of luciferase (e.g., firefly luciferase and Renilla luciferase), green fluorescent protein (GFP), red fluorescent protein (RFP), yellow fluorescent protein (YFP), chloramphenicol acetyltransferase (CAT), alkaline phosphatase (AP) (e.g., secreted embryonic alkaline phosphatase (SEAP)), β-galactosidase (β-gal), β-lactamase (Bla), horseradish peroxidase (HRP), and a variant thereof.

In some embodiments, the genetic locus comprises a gene selected from the group consisting of UCP1, COX7A1, and CIDEA. In some embodiments, the expression of the reporter gene is increased by at least about 2, 5, 10, 20, 40, 60, 80, 100, 120, 140, 160, 180, 200, 250, 300, 400, 500 fold or more, e.g., upon stimulation, e.g., by cold or pathways that elevate cyclic adenosine monophosphate (cAMP). In some embodiments, the cell is derived (e.g., isolated) from the stromal-vascular fraction (SVF) of a subject.

In still another aspect, the disclosure features a method of evaluating a treatment, e.g., a compound, e.g., identifying a therapeutic agent, the method comprising: providing a beige cell described herein, e.g., a beige cell comprising a reporter gene (e.g., a reporter gene described herein), wherein the reporter gene is under the control of, e.g., inserted at, e.g., the transcription of which is initiated from, a genetic locus comprising a gene described herein, e.g., the expression of which is up upregulated upon stimulation, e.g., by cold or pathways that elevate cyclic adenosine monophosphate (cAMP); contacting a compound, e.g., a therapeutic agent, with the cell; and determining the activity of the reporter gene expression product, thereby evaluating a treatment.

In some embodiments, the method further comprises selecting a compound, e.g., a therapeutic agent, that increases the activity of the reporter gene expression product by at least about 2, 5, 10, 20, 40, 60, 80, 100, 200, 400, 600, 800, or 1000 fold, compared to a control, e.g., the activity of the reporter gene expression product when the cell does not contact the compound, e.g., the therapeutic agent.

In some embodiments, the method is performed in vitro. In some embodiments, the method is performed in vivo, e.g., using an animal model of obesity and/or diabetes. In some embodiments, the method further comprises retesting the identified treatment. In some embodiments, the method is performed in vitro, and further comprises retesting in vivo, e.g., using an animal model of obesity and/or diabetes.

### DETAILED DESCRIPTION

Described herein are fat cells that contain a reporter gene. For example, such fat cells can be beige cells or beige progenitor cells. Also described herein are methods that can be used to evaluate treatments, e.g., to identifying compounds (e.g., therapeutic agents), to treat a disorder described herein, such as obesity and diabetes.

Brown Adipose Tissue (BAT)

The cells and methods described herein can be used to augment human brown adipose tissue (BAT) for the treatment of metabolic diseases such as obesity and diabetes. Humans possess activatable BAT as well the precursor cells that would allow further augmentation through cell recruitment. Therapeutic targets and intervention can be designed based on the molecular mechanisms regulating BAT differentiation and activity. Further, targeted genetics and other strategies in animal models can be used to evaluate a treatment that not only augments this tissue, which results in weight loss as predicted by thermodynamic models of energy expenditure, but also has a greater than expected impact on parameters relevant to diabetes. Further, BAT is unlikely to result in toxicology which leads to uncoupling throughout all body organs because of the unique characteristics of this organ and its evolutional purpose.

BAT is capable of adaptive thermogenesis because of the presence of uncoupling protein 1 (UCP1) in its numerous mitochondria. BAT dissipates caloric energy as heat in response to stimulation by the sympathetic nervous system (Cinti S., Nutr. Metab. Cardiovasc. Dis. 2006; 8: 569-574).

A relatively small amount of BAT is present in most humans (typically varying between 9 and 90 grams). Nevertheless, BAT is an organ specifically evolved to safely dissipate energy through the oxidation of glucose and other fuels (Cannon and Nedergaard, Physiol. Rev. 2004; 84: 277-359). Thus, augmentation of BAT levels and/or activity can increase energy expenditure and therefore protect against metabolic disease in our current environment of hyper-nutrition and hypo-activity.

A good portion of the energy expended by BAT comes from the direct oxidation of blood glucose. This property of BAT can be used to observe BAT in humans through imaging technologies. When using positron emission tomography (PET) with 2-deoxy-2-[18F]fluoro-Dglucose ([18F]FDG) tracer combined with computed tomography (CT) for diagnostic imaging of metabolically active tumors and metastases, a symmetrical pattern of increased metabolism is often observed in the supraclavicular region (Nedergaard et al., Am. J. Physiol. Endocrinol. Metab. 2007; 293: 444-452; Yoneshiro et al., Obesity 2011; 19: 13-16). On the basis of PET/CT-guided biopsies from healthy subjects, it has been established that this tissue corresponds to metabolically active BAT since it both presents histological features of BAT and expresses mRNA and proteins that are expected for BAT (Virtanen et al., New Engl. J. Med. 2009; 360: 1518-1525).

Augmentation of BAT levels and/or activity can be achieved by regulation of certain biological pathways (e.g., PRDM16 pathway) that play a role in brown fat cell fate. For example, PRDM16 is a transcription factor and can control brown fat determination (Seale et al., Cell Metab. 2007; 6: 38-54; Seale et al., Nature 2008; 454: 961-967). When PRDM16 is expressed under the control of the fat specific AP2 promoter a significant conversion of subcutaneous white adipose tissue to BAT takes place (Seale et al., J. Clin. Inv., 2011; 121: 96-105). Foxc2 is another transcription factor also known to have profound effects on BAT. When this gene is expressed under the control of the AP2 promoter the whole body fat mass was partially reduced in the FOXC2 Tg mice fed a high-fat diet (Kim et al., Diabetes 2005; 54: 1658-1663). FOXC2 Tg mice were completely protected from diet-induced insulin resistance and intramuscular accumulation of fatty acyl CoA. Further, some other proteins that can increase BAT levels and consequently have positive impacts on metabolic disease include, e.g., BMP7 and MyoK1 (Tseng et al., Nature 2008; 454: 1000-1004; Schulz et al., Proc. Natl. Acad. Sci (USA) 2011; 108: 143-148). Thus, compounds (e.g., therapeutic agents) can be screened based on their abilities to regulate (e.g., activate) the pathways specific to the brown fat cell fate and lineage. Such pathways include, e.g., the PRDM16 pathway or FOXC2 pathway.

Isolation of Beige Cells

In mice, there are at least two distinct types of brown fat tissue. Classical brown fat comes from a myf-5, muscle-like cellular lineage. In addition, UCP1-positive, multilocular cells can emerge in the white adipose tissues under prolonged cold or β-adrenergic agonist exposure. These cells are not derived from the myf-5 lineage and have been named beige cells or brite cells.

Primary stromal vascular fraction (SVF) from inguinal and interscapular depots of mice (e.g., 7-9 week old 129SVE mice (Taconic)) can be fractioned and cultured as described, e.g., in Seale P. et al., J. Clin. Invest. 2011; 121(1):96-105. Primary SVF cells can be cultured in DMEM/F-12 GlutaMax™ (Invitrogen) containing 15% FBS (Gemini Bio-Products, Benchmark). Confluent cultures of clonal lines can be exposed to induction DMEM/F-12 GlutaMax™ (Invitrogen) containing dexamethasone (5 μM), insulin (0.5 μg ml⁻¹), isobutylmethylxanthine (0.5 mM), rosiglitazone (1 μM), triiodothyronine (T3) (1 nM) and 10% FBS, for the purpose of adipocyte differentiation. Four days after induction, cells can be maintained in media containing insulin (0.5 μg ml⁻¹), (T3 (1 nM), and 10% FBS until they are ready for collection. To stimulate thermogenesis, cells can be incubated with 10 μM forskolin for 4 hours, or 10 μM isoproterenol for 6 hours.

Primary SVF cells can be immortalized with 3T3 protocol as described, e.g., in Todaro G. J. and Green H., J. Cell Biol. 1963; 17: 299-313. Limiting dilution of cells into 96-well plates can be used to derive subclones of parental immortalized SVF. Cells can be seeded in a total of forty 96-well plates at a density of 0.2 cells per well. Wells containing cells at approximately 70% confluence can be trypsinized and further propagated in 48-well, then 12-well and finally 6-well dishes. Clonal lines originated from inguinal depots can be initially screened for adipogenic potential after induction. The most adipogenic lines can be selected for further analysis.

Beige cells can be identified based on the expression of one or more of the beige-selective genes. Exemplary beige-selective genes include, but not limited to, CD137.

The expression of beige selective genes can be detected by, e.g., quantitative RT-PCR and Western blot analysis.

For quantitative RT-PCR analysis, total RNA can be isolated from cells by TRIzol (Invitrogen) extraction and purification using QIAGEN RNeasy mini columns according to the manufacturer's instruction. 1 μg of total RNA can be reverse transcribed and analyzed using Applied Biosystems Real-time PCR System using the ΔΔCt method. Relative gene expression can be normalized to TATA box-binding protein (tbp) mRNA levels.

For Western blot analysis, cells can be lysed in RIRP buffer (1% NP-40, 0.5% sodium deoxycholate, 150 mM NaCl, 50 mM Tris-Cl, pH7.5, 0.1% SDS with protease and phosphatase inhibitors). Lysates can be resolved by SDS-PAGE, transferred to PVDF membrane (Millipore), and probed with antibodies.

Total RNA can be isolated from cell lines and used for microarray analysis. Cells can be fully differentiated and treated with 10 μM forskolin for 4 hours before harvested. Array hybridization and scanning can be performed using Affymetrix Gene Chip Mouse

Genome 430 2.0 arrays according to established methods. Microarrays can be first normalized by quantile normalization (Irizarry R. A. et al., Biostatistics 2003; 4: 249-264). To find molecular subentities of beige fat cells within the samples, the established class finding algorithm ISIS, which searches for bipartitions of the unlabeled samples into two groups based on a subset of coherently expressed genes, can be used (von Heydebreck A. et al., Bioinformatics 2001; 17 Suppl 1, S107-14). ISIS can be applied to the data set of inguinal lines, and the search space can be restricted to the pre-filtered genes with the top 1,000 highest gene expression variance. Once the splits are identified, the differentially expressed gene sets can be determined using unpaired t-test at a FDR≦0.01 with fold change greater than 1. To assess the similarity between identified sub-entities with brown fat lines, the brown adipose tissue (BAT) samples can be placed into the dataset and the samples can be hierachically clustered using complete linkage of differentially expressed genes. Principle component analysis can be performed to visualize subtype distributions. Analysis can be performed using the R Software and the Bioconductor (Gentleman R. C. et al., Genome Biol., 2004; 5, R80).

Human beige cells or brown fat lineage cells can be isolated with analogous methods.

Reporter Genes

As used herein, a “reporter” gene encodes any protein that is useful for monitoring gene expression. The reporter can itself be detected; or a product produced or catalyzed by the reporter can be detected; or the reporter can be a change in a property of the cell, e.g., growth, morphology, viability, and the like. For example, the reporter gene can encode a protein which luminesces or fluoresces, which is colored or produces a colored substrate or an enzymatic activity, e.g., phosphatase activity. Exemplary reporter proteins include, e.g., luciferase proteins, fluorescent proteins, chemiluminescent proteins, proteins that can be detected by immunostaining, proteins that can be identified by their enzymatic activity, and radioactively-labeled proteins. The reporter gene is heterologous to the control region under which it is placed.

Exemplary luciferase proteins include, e.g., firefly luciferase and Renilla luciferase. In luminescent reactions, light is produced by the oxidation of a luciferin. Photon emission can be detected by light sensitive apparatus such as a luminometer or modified optical microscopes.

Exemplary fluorescent proteins include, e.g., enhanced blue fluorescent protein (EBFP), enhanced blue fluorescent protein-2 (EBFP2), mKATE, iRFP (infrared fluorescent protein), enhanced yellow fluorescent protein (EYFP), yellow fluorescent protein (YFP), red fluorescent protein (RFP), Katushka, Ds-Red express, TurboRFP, TagRFP, green fluorescent protein (GFP), blue fluorescent protein (BFP), cyan fluorescent protein(CFP), enhanced green fluorescent protein (EGFP), AcGFP, TurboGFP, Emerald, Azami Green, ZsGreen, Sapphire, T-Sapphire, enhanced cyan fluorescent protein (ECFP), mCFP, Cerulean, CyPet, AmCyan1, Midori-Ishi Cyan, mTFP1 (Teal), Topaz, Venus, mCitrine, YPet, PhiYFP, ZsYellow1, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143. Fluorescent proteins can be assayed, e.g., by FACS or fluorescence microscopy.

Other exemplary reporter proteins include beta-galactosidase (encoded by the lacZ gene), any polypeptide comprising a detectable protein tag, such as a FLAG tag or HISx6 tag, a c-myc tag or a HaloTag® (Promega Corporation).

Reporter gene expression can be assayed by immunohistochemistry, e.g., by detecting expressed proteins with antibodies labeled with different detectable probes (e.g., Alexa fluor®, Oregon Green® or Pacific Blue®; horseradish peroxidase (HRP) and alkaline phosphatase (AP)). In one embodiment, beta-galactosidase is assayed using X-Gal substrate.

Gene Targeting and Production of Genetic Engineered Cells

Methods for gene targeting and producing genetic engineered cells are known in the art, e.g., as described in Morrow B. and Kucherlapati R., Curr. Opin. Biotechnol. 1993; 4(5):577-82; Hanson K. D. and Sedivy J. M., Mol. Cell Biol. 1995; 15(1):45-51; Willnow T. E. and Herz J., Methods Cell Biol. 1994; 43 Pt A: 305-34; and Waldman T. et al., Curr. Protoc. Mol. Biol. 2003; Chapter 9: Unit 9.15.

Animal Models and Testing A suitable in vivo animal model (e.g., mouse model) can be used to evaluate the effect of the identified treatment, e.g., a compound, e.g., a therapeutic agent, on treating disorders such as obesity and diabetes. A suitable animal model (e.g., mouse model) of obesity and/or diabetes is known in the art and can be obtained or rendered by feeding C57BALB/c mice a high fat diet containing a high percentage of calories from fat. Mice are administered with an identified compound (e.g., therapeutic agent), e.g., by injection (e.g., intravenous injection), or oral administration, and the expression and activity of the compound (e.g., therapeutic agent) can be analyzed at predetermined time points post administration, e.g., 5 days, 7 days, 10 days, 14 days, 21 days, etc; post administration. The level of the compound (e.g., therapeutic agent) can be analyzed in the mouse plasma derived from tail blood sampling and/or in the mouse fat pads post mouse sacrifice, collection, and processing of the fat pad tissue. Methods of measuring the level of the compound (e.g., therapeutic agent) are known in the art, e.g., HPLC, MS, ELISA, and qRT-PCR. Experimental results can be compared to a control group of mice administered a suitable control compound.

To evaluate the effect of a treatment (e.g., a compound, e.g., a therapeutic agent) on mouse weight and body fat parameters, mouse body weight can be measured using a suitable scale; and analysis of body fat content can be measured using a dual energy X-ray absorptiometry (DEXA) scanner, both utilizing standard protocols known to those skilled in the art. Whole body NMR, MRI, and X-ray computer topography (e.g., CT and micro-CT) can also be performed.

Diabetes (e.g., diet induced insulin resistant diabetes) can be associated with impaired insulin and glucose tolerance as well as elevated levels of fasting plasma insulin and glucose concentrations. In order to evaluate the effect of a treatment (e.g., a compound, e.g., a therapeutic agent) on these metabolic parameters, standard metabolic tests known to those skilled in the art can be performed using standard techniques. In vivo insulin tolerance can be tested using a standard insulin tolerance test known to those skilled in the art. Briefly, mice fasted for several hours are injected intraperitoneally with insulin at approximately 0.8 U/kg. Fasting plasma insulin concentrations can be measured using a standard insulin enzyme linked immunosorbant assay (ELISA) using plasma derived from tail blood sampling at specific time points pre and post insulin administration. In vivo glucose tolerance can be tested using a standard glucose tolerance test known to those skilled in the art. Briefly, mice fasted for several hours are injected intraperitoneally with glucose at approximately 2 g/kg. Fasting plasma glucose concentrations can be measured using a standard glucometer using plasma derived from tail blood sampling at specific time points pre and post glucose administration. Insulin resistance is indicated by elevated fasting plasma insulin and fasting plasma glucose concentrations, as well as impaired glucose and insulin tolerance. The effect of a treatment (e.g., a compound, e.g., a therapeutic agent) on glucose uptake in vivo can be analyzed by positron emission tomography (PET) with fluorodeoxyglucose (¹⁸FDG), using standard PET scan techniques previously published and known to those skilled in the art. Active brown adipose tissue functions as a repository for glucose disposal and thus features increased levels of glucose uptake. PET measures glucose uptake, and can thus be used to detect functioning brown adipose tissue in vivo. PET scans can be conducted on the mice or tissue derived from the mice using standard PET scan techniques previously published and known to those skilled in the art.

The effect of a treatment (e.g., a compound, e.g., a therapeutic agent) on oxygen consumption can be measured using Comprehensive Lab Animal Monitoring System (CLAM) cages. The principal setup of the CLAM based respirometric system has been previously published and is known to those skilled in the art. Briefly, volumes of oxygen consumed and volumes of carbon dioxide produced by each mouse can be measured at specific time intervals using electrochemical oxygen and carbon dioxide analyzers. Oxygen and carbon dioxide readings can further be converted to metabolic rate.

The effect of a treatment (e.g., a compound, e.g., a therapeutic agent) on energy intake in vivo can be measured by the level of food consumption. The effect of a treatment (e.g., a compound, e.g., a therapeutic agent) on energy expenditure in vivo can be measured using several techniques known to those skilled in the art. Energy expenditure can be a measure of oxygen consumption, measured as described above; physical activity, which can be simultaneously, measured using the CLAM cages using standard protocols known to those skilled in the art; and/or thermogenesis, measured as described below.

The effect of a treatment (e.g., a compound, e.g., a therapeutic agent) on in vivo thermogenic capacity can be examined by several techniques, known to those skilled in the art. Shivering thermogenesis can be evaluated by conducting a standard cold challenge assay, using standard protocols known to those skilled in the art. Briefly, mice can be exposed to a below thermoneutral temperature, e.g., 4° C., and their subsequent shivering response and core body temperature monitored at several predetermined time points pre and post the start of cold exposure. The mice can be analyzed for the display of signs of insensitivity to the cold, e.g., the ability to keep their body temperature around baseline; or sensitivity to the cold, e.g., a sustained drop in body temperature or hypothermia or lethal hypothermia. The effect of a treatment (e.g., a compound, e.g., a therapeutic agent) on mouse thermogenesis can also be measured by collecting brown fat tissue after several predetermined time points pre and post cold exposure and analyzing the gene expression of genes associated with mouse thermogenesis and brown fat cell thermogenesis, e.g., UCP1, PGC1α.

The effect of a treatment (e.g., a compound, e.g., a therapeutic agent) on mouse non-shivering thermogenesis can be evaluated by conducting a norepinephrine injection test, using standard protocol known to those skilled in the art. Briefly, mice acclimated to a below thermoneutral temperature, e.g., 4° C., are injected with norepinephrine, and maintained in the below thermoneutral temperature, e.g., 4° C., while temperature and oxygen consumption is measured as described above at several predetermined time points pre and post norepinephrine administration. The effect of a treatment (e.g., a compound, e.g., a therapeutic agent) on mouse non-shivering thermogenesis can also be measured by collecting brown fat tissue after several predetermined time points post epinephrine administration and analyzing the gene expression of genes associated with mouse thermogenesis and brown fat cell thermogenesis, e.g., UCP1, PGC1α.

Kits

Cells, compounds, and reagents required to practice the claimed methods can be provided in a kit. The kit can include (a) a container that contains a cell described herein; optionally (b) reagents and buffers for use with the cell, e.g., for evaluating a treatment; and optionally (c) informational material.

The informational material can be descriptive, instructional, marketing or other material that relates to the methods described herein and/or the use of the cells for evaluating a treatment, e.g., a compound, e.g., a therapeutic agent. The informational material can include information about one or more of: providing a cell described herein, e.g., a beige cell comprising a reporter gene, wherein the reporter gene is under the control of, e.g., inserted at, a genetic locus comprising a gene described herein; contacting a compound, e.g., a therapeutic agent, with the cell; and determining the activity of the reporter gene expression product, e.g., to arrive at a conclusion regarding evaluation of a treatment.

The informational material of the kits is not limited in its form. The information can be provided in a variety of formats, including printed text, computer readable material, video recording, or audio recording, or information that provides a link or address to substantive material. The informational material can be provided on a label, e.g., on a vial comprising a cell.

In addition to a cell, the composition in the kit can include other ingredients, such as a solvent or buffer, a stabilizer, or a preservative. The components of the kit can be provided in any form, e.g., a liquid, dried or lyophilized form, and substantially pure and/or sterile. When the agents are provided in a liquid solution, the liquid solution is typically an aqueous solution. When the agents are provided as a dried form, reconstitution generally is by the addition of a suitable solvent. The solvent, e.g., sterile water or buffer, can optionally be provided in the kit.

The kit can include one or more containers. The kit can contain separate containers, dividers or compartments for the composition and informational material. For example, a composition comprising a cell, can be contained in a bottle or vial or tube, and the informational material can be contained in a plastic sleeve or packet. The separate elements of the kit can also be contained within a single, undivided container. For example, the composition is contained in a bottle or vial or tube that has attached thereto the informational material in the form of a label. The kit can include a plurality (e.g., a pack) of individual containers, each containing one or more unit dosage forms (e.g., a dosage form described herein) of the agents. The containers of the kits can be air tight, waterproof (e.g., impermeable to changes in moisture or evaporation), and/or light-tight.

What is claimed is:

1. A beige cell comprising a reporter gene wherein the reporter gene is under the control of a gene in the PRDM16 pathway.

2. The beige cell of claim 1, wherein said gene is PRDM16.

3. The beige cell of claim 1, wherein the cell is, a human cell or a mouse cell.

4. The beige cell of claim 1, wherein the cell is a primary cell, a non-primary cell, an immortalized cell, or a clonal cell.

5. The beige cell of claim 1, wherein the reporter gene encodes a protein which luminesces or fluoresces, which is colored or produces a colored substrate or an enzymatic activity.

6. The beige cell of claim 1, wherein the reporter gene encodes a protein selected from the group consisting of luciferase, green fluorescent protein (GFP), red fluorescent protein (RFP), yellow fluorescent protein (YFP), chloramphenicol acetyltransferase (CAT), alkaline phosphatase (AP), β-galactosidase (β-gal), β-lactamase (Bla), horseradish peroxidase (HRP), and a variant thereof.

7. The beige cell of claim 1, wherein the genetic locus is selected from PRDM16, PGC-1α, and PGC-1β.

8. The beige cell of claim 1, wherein the cell is derived from the stromal-vascular fraction (SVF) of a subject.

9. A method of evaluating a compound, the method comprising:
providing a beige cell of claim 1;
contacting a compound the cell; and
determining the activity of the reporter gene expression product; thereby evaluating a compound.

10. The method of claim 9, further comprising selecting a compound that increases the activity of the reporter gene expression product by at least about 2, 5, 10, 20, 40, 60, 80, 100, 200, 400, 600, 800, or 1000 fold, compared to a control.

11. The method of claim 9, wherein the method is performed in vitro.

12. The method of claim 9, wherein the method is performed in vivo.

13. The method of claim 9, further comprising retesting the evaluated compound.

14. The method of claim 9, wherein the method is performed in vitro, further comprising retesting in vivo.

15. The method of claim 14, wherein the retesting is in an animal model of obesity and/or diabetes.

16. The method of claim 9, further comprising evaluating one or more genes, the expression of which is upregulated upon stimulation, by cold or pathways that elevate cyclic adenosine monophosphate (cAMP).

17. A beige cell comprising a reporter gene, wherein the reporter gene is under the control of a gene, the expression of which is upregulated upon stimulation by cold or pathways that elevate cyclic adenosine monophosphate (cAMP).

18. A method of evaluating a compound, the method comprising:
providing a beige cell of claim 17;
contacting a compound with the cell; and
determining the activity of the reporter gene expression product; thereby evaluating a compound.

19. The method of claim 18, wherein the method is performed in vivo.

20. The method of claim 18, further comprising retesting the evaluated compound.
